(19) United States
(12) Patent Application Publication
(10) Pub. No.: US 2019/0023852 A1
(43) Pub. Date: Jan. 24, 2019

(54) BRANCHED POLYESTER CARRYING DENDRONS

(71) Applicant: The University of Liverpool, Liverpool (GB)

(72) Inventors: Steven Rannard, Merseyside (GB); Andrew Owen, Merseyside (GB); Pierre Chambon, Merseyside (GB); Hannah Rogers, Merseyside (GB); Sarah Blackmore, Merseyside (GB)

(21) Appl. No.: 16/068,536

(22) PCT Filed: Dec. 29, 2016
(86) PCT No.: PCT/GB2016/054084

§ 371 Date: Jul. 6, 2018

(30) Foreign Application Priority Data
Jan. 7, 2016 (GB) 1600289.1

(51) Int. Cl. C08G 83/00; A61K 47/34

(52) U.S. Cl. CPC C08G 83/003; A61K 47/34

(57) ABSTRACT

Branched polyesters carrying dendrons are a useful class of nanomaterials which exhibit good handling properties and stability, can degrade to a high extent, and are effective encapsulation materials. They can be used to make nanoprecipitated particles which may for example be used in therapy. Furthermore, these materials can be synthesised by economical and tailorable processes. The materials can be prepared by the ring-opening polymerisation (ROP) of mono-functional lactone monomers and difunctional lactone monomers, using dendron initiators.

The present invention relates to polymer architectures which contain dendrons. Such structures are hybrid materials, containing polymeric parts and dendritic parts, and some classes of such materials are also known as polydendrons.

Dendrimers have been extensively studied in the context of medical and other applications. The word “dendrimer” was coined in the early 1980s, following work on cascade chemistry and arborols, to describe polymers which contain dendrons. A “dendron” is a tree-like, repeatedly-branched, moiety. Thus, a dendron is a wedge-shaped dendritic fragment of a dendrimer. Typically, dendrimers have ordered, symmetrical architectures. A dendrimer comprises a core from which several dendrons branch outwards, to form a three-dimensional, usually spherical structure.

Dendrimers can be prepared by step-wise divergent or convergent growth. Divergent procedures start at the core of the dendrimer and grow outwards. Convergent procedures prepare dendrons first and then couple the dendrons together. In convergent procedures, the dendrons are typically coupled together at their focal points (i.e. at the base of the “tree”, or the apex of the dendritic wedge) via chemically addressable groups.

Many of the interesting and useful properties of dendrimers arise from their multivalency. By analogy with a tree having many leaves, a dendrimer terminates in many moieties. Due to their repeatedly branched iterative nature, they are large compared to non-polymeric active molecules and contain a large number of surface groups, and can therefore encapsulate, and/or be conjugated to, a large amount of material.

Further description of dendrimers and their structures, preparation and applications, can be found in numerous articles including: S. M. Grayson and J. M. Fréchet, Chem. Rev. 2001, 101, 3819-3867; H. Frauenrath, Prog. Polym. Sci 2005, 325-384; F. Aulenta, W. Hayes and S. Rannard, European Polymer Journal 2003, 39, 1741-1771; E. R. Gillies and J. M. J. Fréchet, Drug Discovery Today, 2005, 10, 1, 35-43; and S. H. Medina and M. E. H. El-Sayed, Chem. Rev. 2009, 109, 3141-3157.

Whilst dendrimers have many interesting properties and promising features, they also have significant disadvantages. Dendrimer syntheses are lengthy and costly. The production of ideally branched structures requires multiple repeated steps of synthesis, purification and characterisation. Maintaining a 100% degree of branching generates complexity and takes time and requires very controlled reaction conditions. Even with high levels of successful recovery between steps, the compound effect after several steps means that the overall mass recovery suffers significantly. Whilst convergent methods are better than divergent methods from the viewpoint of ease and speed of procedure, they are still arduous, and other problems beset convergent methods, for example steric difficulties hindering coupling.

Geometric realities of iterative branching mean that the crowding constraints at the surface of the dendrimer sphere limit the size of the nanomaterials. Therefore dendrimers typically have a maximum size of about 10 nm. This limits the amount of material they can carry.

Polydendrons, such as those disclosed in WO 2009/122220, WO 2014/199174 and WO 2014/199175, comprise branched vinyl polymer scaffolds carrying dendrons, and possess advantageous dendrimer-type properties, in part due to their multiply-branched nature, without the disadvantages of complex conventional dendrimer processes. Further publications regarding polydendrons include: H. E. Rogers, P. Chambon, S. E. R. Auty, F. Y. Hem, A. Owen and S. P. Rannard, Soft Matter 2015, 11, 7005-7015; F. L. Hatton, L. M. Tatham, L. R. Tidbury , P. Chambon, T. He, A. Owen and S. P. Rannard, Chem. Sci. 2015, 6, 326-334; and F. L. Hatton, P. Chambon, T. O. McDonald, A. Owen and S. P. Rannard, Chem. Sci. 2014, 5, 1844-1853.

Such polydendrons, however, are only suitable for use in certain scenarios, and there is a need for alternative types of polydendron to enhance the applicability of this area of technology. Furthermore, whilst the previously disclosed polydendrons are highly effective, polydendrons with improved properties would be advantageous.

We have carried out further research in this field and have found that certain aspects of polydendron syntheses and chemistry are highly unpredictable.

However, we have now found that a further class of polydendron materials can be reliably synthesised and exhibit useful properties.

Therefore, from a first aspect, the present invention provides a branched polyester carrying dendrons.

We have found that such material represents a useful class of nanomaterials which exhibit good handling properties and stability, can degrade to a high extent, and are effective encapsulation materials. They can be used to make nanoprecipitated particles which may for example be used in therapy. Furthermore, these materials can be synthesised by economical and tailorable processes.

The branched polyester carrying dendrons can be considered to comprise a “scaffold” (the branched polyester) to which dendrons are covalently bonded. Thus a plurality of dendrons are present, without requiring the cost, complexity, or arduous synthesis of dendrimers.

The scaffold or core comprises polyester chains linked by branches. Optionally, the polyester chains may have between 1 and 6 carbon atoms between ester linkages. For example, the polyester chains may have 5 carbon atoms between ester linkages, or 1 carbon atom between ester linkages, or different numbers of carbon atoms between linkages.

Optionally, each branch between the chains may be a single covalent bond, or may comprise between 1 and 6 carbon atoms, or may comprise other linkages, for example ether, ester or amide linkages.

The branched polyester may be made from a monofunctional lactone monomer and may be branched by virtue of a difunctional lactone monomer.

The term lactone, herein, denotes a cyclic ester, in other words a compound wherein an ester linkage is present as part of a ring. More than one ester linkage may be present as part of the ring. Thus the term lactone, herein, also encompasses cyclic di-esters, for example lactide or glycolide.

The lactone monomer may for example be ε-caprolactone, lactide, glycolide, or a mixture of lactide and glycolide. The brancher may for example be BOD (4,4′-bioxepanyl-7-7′-dione). Structures are shown below. Optionally the lactone monomers and/or branchers may be substituted or functionalised. Alternatively, other lactones, cyclic di-esters and/or other branchers may be used.

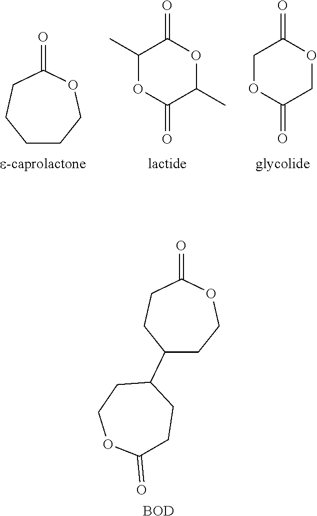

The monofunctional lactone monomer (e.g. ε-caprolactone) reacts to form a polyester chain by ring-opening polymerisation (ROP) of one ring. The difunctional lactone monomer (e.g. BOD) has two rings which are bonded together such that each ring can open and become part of a polyester chain, and such that the bond or linker between the two rings becomes a bridge between polymer chains, thereby bringing about the branched polyester structure.

Ring opening polymerisation methods and materials are known in the art, for example from Nguyen et al, Polym Chem 2014, 5, 2997-3008. This document discloses a tin octanoate—catalysed method. The ring opening polymerisation in the present invention may be carried out using organometallic catalysis (e.g. with tin octanoate) or in other ways (e.g. using acid catalysis, e.g. using trifluoroacetic acid).

The dendrons may be incorporated by using dendron initiators.

Thus, from a further aspect the present invention provides a method of preparing a branched polyester carrying dendrons, comprising ring-opening polymerisation

(ROP) of a monofunctional lactone monomer and a difunctional lactone monomer, using a dendron initiator.

A functional group, for example a primary alcohol, may be present at the focal point of a dendron, and may be used to initiate the ROP.

One or more than one type of lactone may be used. For example, whereas lactide may be polymerised to form PLA, and glycolide may be polymerised to form PGA, a mixture of lactide and glycolide may be polymerised to form PLGA [poly(lactic-co-glycolic acid) or poly(lactide-co-glycolide)]. PLGA 75:25, for example, denotes 75% lactide and 25% glycolide (molar ratio). Other monomer combinations are also possible: for example we have copolymerised ε-caprolactone and lactide to form PCL/PLA copolymers; this may be done using an acid-catalysed method.

Dendron-based initiators can be used with various different types of ROP. For example, metal catalysts can be used, as described in e.g. Arbaoui et al, Polym Chem 2010, 1, 804-826; and cationic ROP with acid catalysis can be carried out, as described in e.g. Bourissou et al, Macromolecules 2005, 38, 9993-9998, Basko et al, Journal of Polymer Science: Part A: Polymer Chemistry 2007, 45, 3090-3097, Basko et al, Journal of Polymer Science: Part A: Polymer Chemistry 2006, 44, 7071-7081, and Gazeau-Bureau et al, Macromolecules 2008, 41, 3782-3784.

One advantage of the present invention is that it provides completely degradable materials. This contrasts with the materials disclosed in WO 2009/122220, WO 2014/199174 and WO 2014/199175: such materials are only degradable if degradable functionality is built into the scaffold, and even then, part of the polymer generally remains connected to each dendron after the scaffold has been broken apart. Furthermore, breaking apart the scaffold in the prior art polydendrons can involve a multi-step process due to their greater stability.

The discussion above relates primarily to the scenario where the scaffold comprises a branched polyester and no other polymer. Alternatively, it is possible in accordance with the present invention for the polymer to contain not only polyester chemistry but also other types of polymer, for example vinyl polymer chemistry. For example, methods for the preparation of products of the present invention may comprise not only ROP (to form polyester parts) but also ATRP or other processes (to form vinyl polymer parts). In some embodiments, macroinitiators may be used (in addition to the dendron initiators) so that one block of a block copolymer may be derived from the macroinitiator and another block of said block copolymer may be formed by polymerisation initiated by the initiator(s).

There is no particular limitation regarding the type of dendron that can be used, or the chemistry used to prepare the dendrons. In some scenarios it is desirable to have particular groups present at the surface (i.e. at the tips of the “branches” of the dendron), and these may be incorporated during the synthesis of the dendron. Any suitable coupling chemistry may be used to build up the dendrons. They may for example contain tertiary amine and ester linkages. Alternatively they may comprise other chemistry.

Some possible dendron initiators which have been used are shown in the examples. These include a first generation dendron initiator (G₁) and a second generation dendron initiator (G₂). It should be noted, however, that these are merely examples and that other dendron initiators may be used.

Post-polymerization functionalization of the dendrons may be carried out, for example to achieve chemistries which are not compatible with ROP.

Optionally, more than one initiator may be used, so long as at least one of the initiators is a dendron initiator. Thus, mixed initiators may be used, as described in WO 2014/199174. In other words, not only a dendron initiator may be used but also one or more further initiator (which may be a different type of dendron initiator, or alternatively an initiator other than a dendron initiator). This allows considerable further advantages in terms of varying the composition and the properties of the resultant polydendron structure, as described in WO 2014/199174. The different initiators are distributed statistically and evenly around the surface of the branched polymer scaffold. Some polymer chains will have one type of initiator at one end whereas other polymer chains will have another type at their end. There may be two types of initiator, or more, e.g. three or four or more, and therefore the multiplicity of types of end group may be two or more. There are synergistic advantages: for example the use of dendrons and other moieties as initiators means that they do not need to be introduced separately but instead are used as reagents within an already very efficient and convenient polymerization process. The process conveniently and cost-effectively results in the different types of initiators being distributed throughout the materials. The initiators themselves are relatively easy to synthesize. The further initiator may alter the properties of the polydendron, for example the solubility, hydrophilicity, hydrophobicity, aggregation, size, reactivity, stability, degradability, therapeutic, diagnostic, biological transport, plasma residence time, cell interaction, drug compatibility, stimulus response, targeting and/or imaging characteristics. Non-dendron initiators may for example comprise polyethylene glycol (PEG) groups.

Polymerisation may be controlled so as to achieve non-crosslinked structures. Controlling the conditions including the amount of initiator(s) and brancher may be used to bring about on average one branch or fewer per polyester chain, or indeed different amounts of branching. The present invention thus allows the preparation of non-gelled products. The solubility and viscosity of the products can be controlled. The present invention allows the preparation of polymer structures which exhibit good solubility and low viscosity in contrast with some polymer structures of the prior art which are insoluble and/or exhibit high viscosity and/or are extensively cross linked in soluble polymer networks, high molecular polymers, or are other materials which exhibit unsuitable properties.

From further aspects the present invention provides various uses of the branched polyesters carrying dendrons. The products may be used to encapsulate or carry, or may be loaded with, various other entities, for example medically useful materials including drugs, pro-drugs, or diagnostically useful materials. These may be used in methods of medical treatment, diagnosis or surgery in respect of subjects, for example humans and other mammals. The invention facilitates controlled or tailored delivery, release and/or degradation. The invention is also useful in non-medical contexts in relation to crosslinking, coating and deposition, for example.

The present invention will now be described, by way of example only, in further non-limiting detail, with reference to the following figures and examples in which:

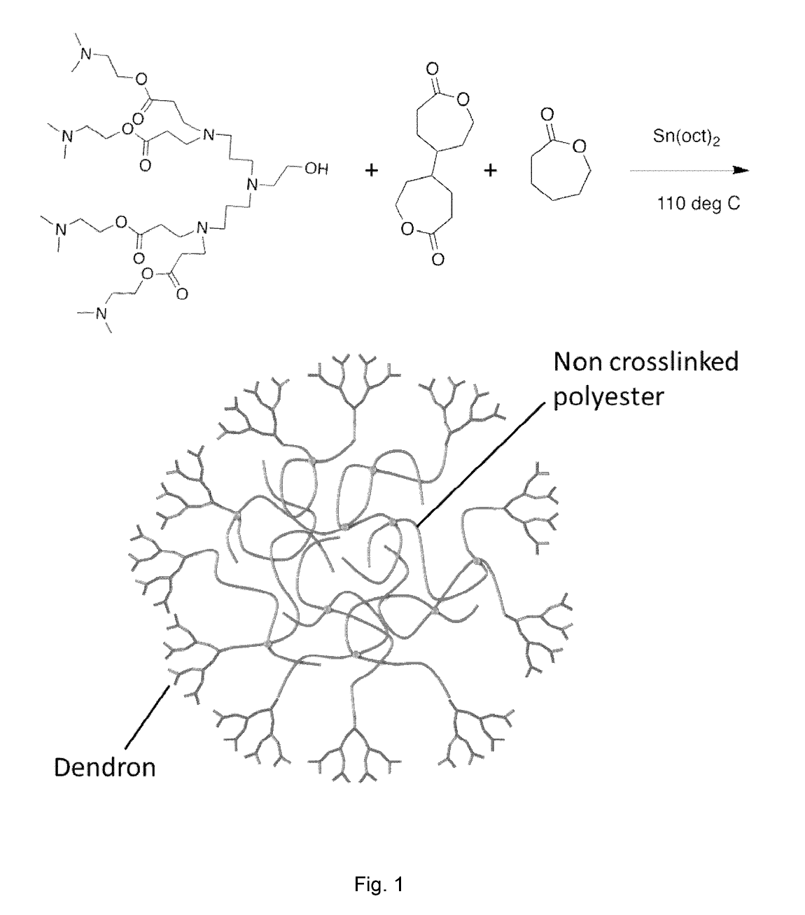

FIG. 1 shows a reaction scheme according to which a dendron initiator may be reacted with a difunctional lactone monomer and a monofunctional lactone monomer to form a polydendron material which comprises a non-crosslinked polyester core carrying a plurality of dendrons;

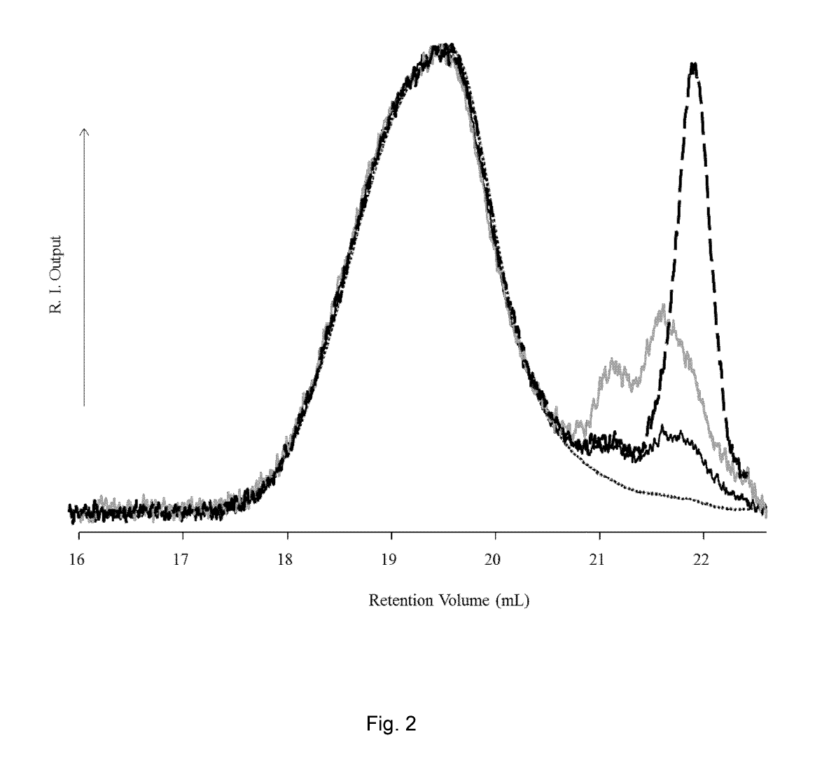

FIG. 2 shows a size-exclusion chromatogram (SEC) demonstrating the reliable degradation of products in accordance with the present invention to low molecular weight materials; and

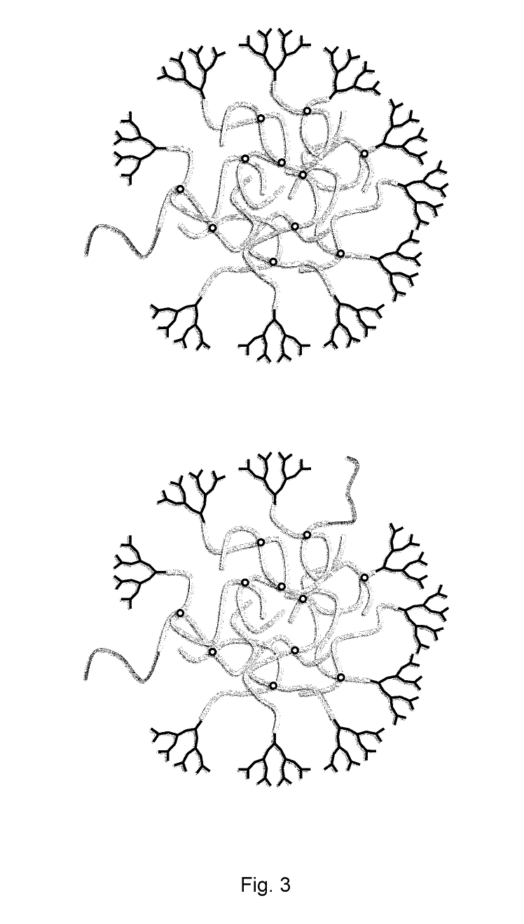

FIG. 3 shows polydendron materials wherein the polymer scaffold carries not only dendrons but also other moieties.

### EXAMPLES

A. Ring Opening Polymerisation of ε-caprolactone using Tertiary Amine Functionalised Dendritic Initiators

1.0 Initiator Synthesis

1.1 Generation 1 (G₁) dendron ROP initiator synthesis

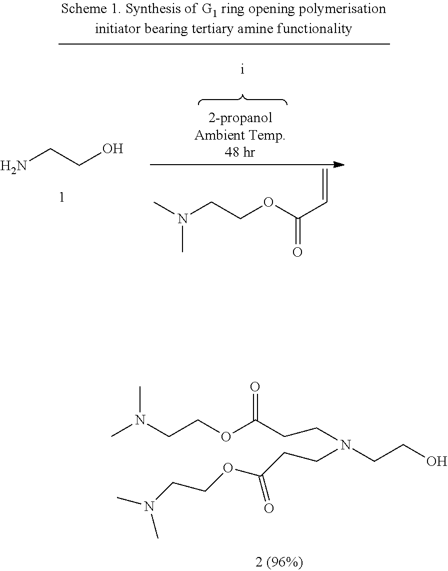

[G₁ dendron ROP initiator]; [2]-2-(Dimethylamino)ethyl acrylate (DMEA) (6.0 g, 42 mmol, 6 eq.) was added to a 50 mL round 2 necked round-bottomed flask containing propan-2-ol (IPA) (12 mL). The flask was deoxygenated under a positive N₂ purge for 10 minutes. Ethanolamine [1] (0.4266 g, 7.0 mmol, 1 eq.) dissolved in IPA (12 mL) was added drop wise while the solution was stirring in an ice bath under a positive flow of N₂. The final mixture was stirred for a further 10 minutes at 0° C. before being allowed to warm to room temperature and left stirring for 48 hr. The solvent was removed and the product left to dry in vacuo overnight. Yield: 2.33 g, yellow oil (96%). ¹H NMR (400 MHz, CDCl₃) δ 2.27 (s, 12H), 2.44-2.61 (m, 10H), 2.81 (t, 4H), 3.57 (t, 2H), 4.18 (t, 4H). ¹³C NMR (100 MHz, CDCl3) δ 32.6, 45.6, 49.4, 56.2, 57.8, 59.5, 62.0, 172.7. Calcd [M+H]⁺ (C₁₆H₃₃N₃O₅) m/z=347.5. Found: ESI-MS: [M+H]⁺ m/z=348.2. Anal. Calcd for C₁₆H₃₃N₃O₅: C, 55.26; H, 9.50; N, 12.09%. Found C, 57.09; H, 9.47; N, 11.02%.

[2] was prepared using the literature procedure: Polymer Chemistry, 2015, 6, 573

1.2 Synthesis of 1-[N, N-bis (2-aminopropyl)-amino]-1-propanol (APAP)

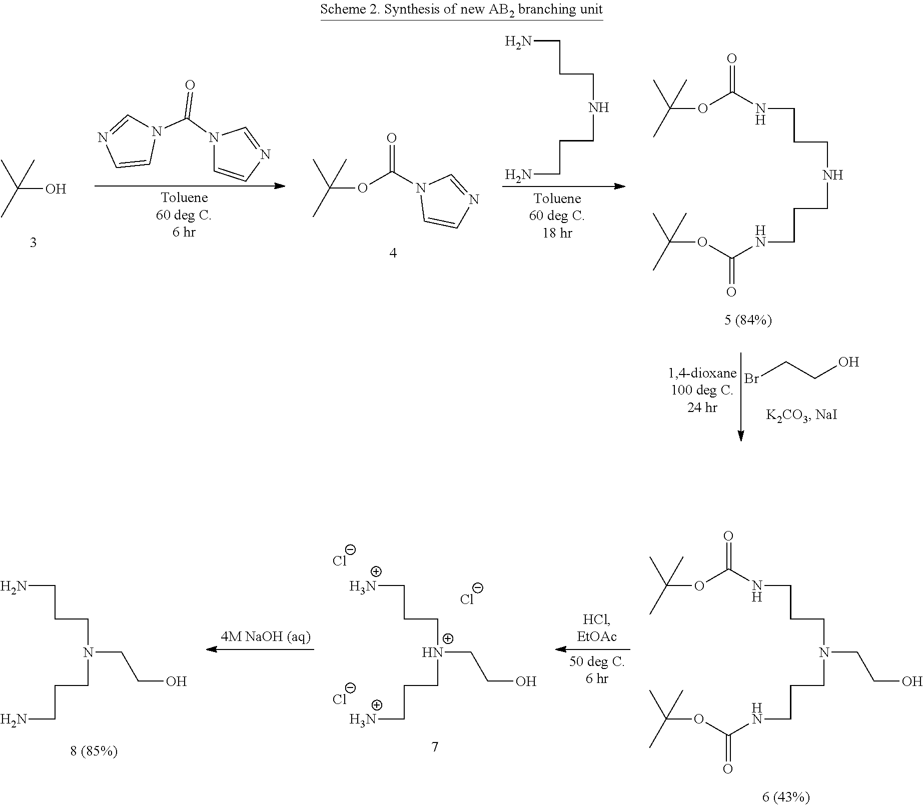

1.2.1 Synthesis of [^{t}BOC₂-BAPA-G1]

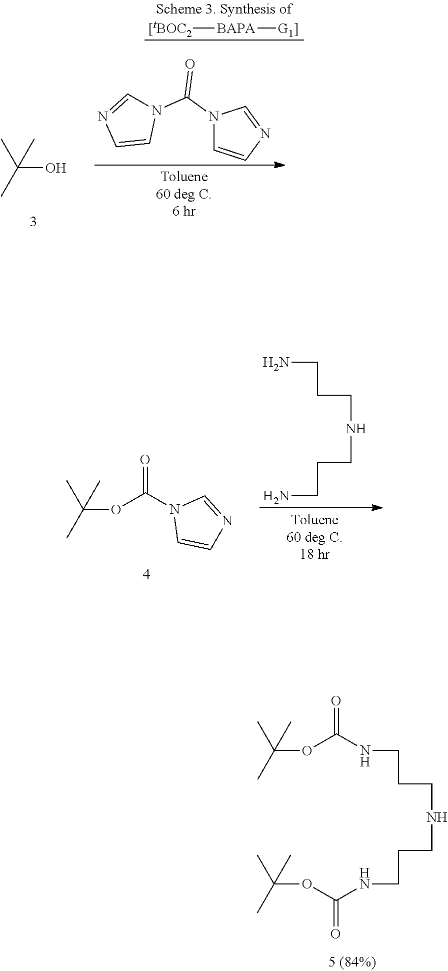

[^{t}BOC₂-BAPA-G₁]; [5]—Carbonyl diimidazole (CDI) (19.55 g, 0.121 mol, 2 eq.) was added to an oven-dried 500 mL 2-neck RBF fitted with a reflux condenser, magnetic stirrer and a dry N₂ inlet. 350 mL of anhydrous toluene was added and the flask purged with N₂ for 10 minutes. The solution was stirred at 60° C. and tertiary butanol

[3] (17.83 g, 23 mL, 0.241 mol, 4 eq.) was added via a warm syringe. The mixture was left stirring at 60° C. for 6 hr under a positive flow of nitrogen. Bis(3-aminopropyl)amine (7.88 g, 8.4 mL, 0.060 mol, 1 eq.) was added dropwise, and the reaction was left stirring for a further 18 hr at 60° C. under a positive flow of nitrogen. Following this, the solution was allowed to cool to room temperature, and the pale yellow solution was filtered to remove any solid imidazole, and concentrated in vacuo. The resulting viscous oil was dissolved in dichloromethane (200 mL) washed with distilled water (3×200 mL) and once with brine (150 mL). The organic layer was dried with anhydrous Na₂SO₄, filtered, and concentrated in vacuo. Yield: 16.63 g, white solid, (84%). ¹H NMR (400 MHz, CDCl₃): δ=1.43 (s, 18H), 1.63 (m, 4H), 2.64 (t, 4H), 3.20 (t, 4H), 5.19 (s, br, NH—). ¹³C NMR (100 MHz, CDCl₃): δ=28.5, 29.9, 39.0, 47.7, 79.2, 156.2. Calcd: [M+H]⁺ (C₁₆H₃₃N₃O₄) m/z=332.3. Found: ESI-MS: [M+H]⁺ m/z=332.3. Anal. Calcd for C₁₆H₃₃N₃O₄: C, 58.00; H, 10.00; N, 12.69. Found: C, 57.78; H, 9.92; N, 12.82.

[5] was prepared using the literature procedure: Soft Matter, 2012, 8, 1096.

1.2.2 Synthesis of [^{t}BOC₂-APAP-pOH]

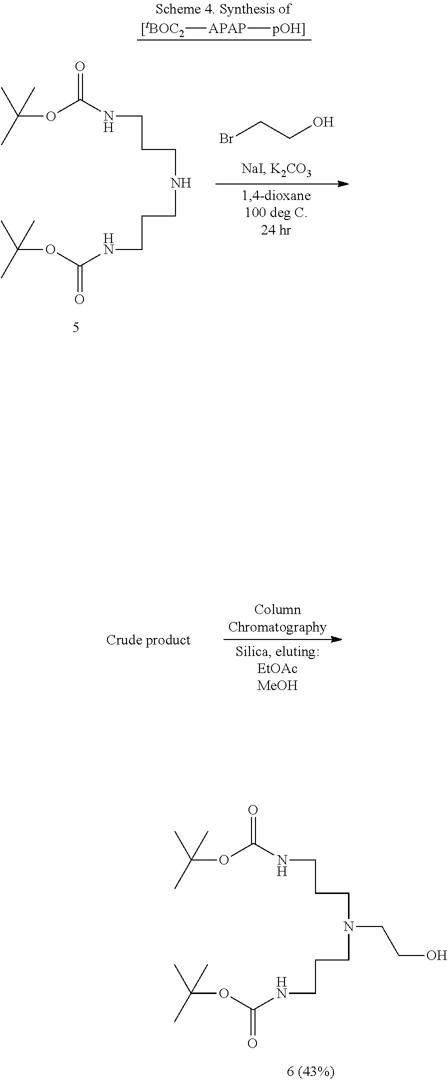

[^{t}BOC₂-APAP-pOH]; [6]-[5] (15.38 g, 0.046 mol, 1 eq.), bromoethanol (5.81 g, 3.3 L, 0.046 mol, 1 eq.), sodium iodide (150 mg), potassium carbonate (19.27 g, 0.139 mol, 3 eq.) and 1,4-dioxane (150 mL) was added to a 500 mL 2-necked RBF fitted with a reflux condenser and magnetic stirrer. The reaction was refluxed overnight. After this time, water (150 mL) was added to the reaction mixture and the product was extracted with ethyl acetate (2×225 mL). The combined extracts were washed with water (1×150 mL), dried over sodium sulfate and concentrated in vacuo. The crude product purified by liquid chromatography (silica gel, eluting with EtOAc:MeOH, 80:20). Yield: 7.55 g, pale yellow oil at ambient temperature, solidifying to an off white solid upon cooling, (43%). ¹H NMR (400 MHz, CDCl₃): δ=1.41 (s, 18H), 1.62 (m, 4H), 2.47 (t, 4H), 2.54 (t, 2H), 2.85 (s, br, OH), 3.16 (m, 4H), 3.57 (t, 2H), 5.09 (s, br, NH). ¹³C NMR (100 MHz, CDCl₃): δ=27.2, 28.4, 38.9, 51.7, 56.0, 58.9, 79.1, 156.2. Calcd: [M+H]⁺ (C₁₈H₃₇N₃O₅) m/z=376.5. Found: ESI-MS: [M+H]⁺ m/z=376.3. Anal. Calcd for C₁₈H₃₇N₃O₅: C, 57.52; H, 9.85; N, 11.18. Found: C, 56.97; H, 9.81; N, 11.02.

[6] was prepared using the literature procedure: Journal of Medicinal Chemistry, 1994, 37 (15), 2334.

1.2.3 Synthesis of 1-[N, N-bis (2-aminopropyl)-amino]-1-propanol (APAP)

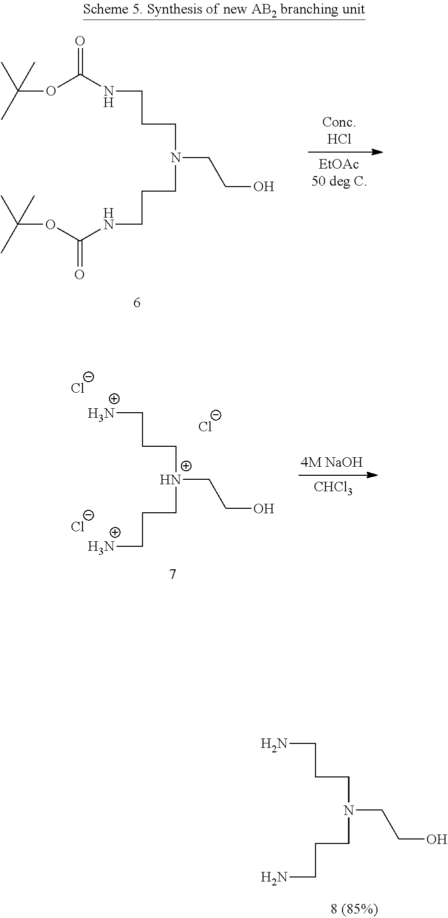

[APAP]; [8]—To a 500 mL RBF, 6 (7.49 g, 0.02 mol, 1 eq.) was dissolved in ethyl acetate (80 mL), and had concentrated HCl (12.14 g, 10.3 mL, 36% active) added very slowly. CO₂ began to rapidly evolve. The reaction vessel was left open to the atmosphere, heated to 50° C. and stirred for 24 hr. After removal of ethyl acetate in vacuo, the crude oil was dissolved very slowly in 4M NaOH (80 mL), and reduced by approximately half its volume on the rotary evaporator (60° C.). A yellow oily substance formed on the surface of the NaOH solution. The mixture was extracted with CHCl₃ (2×80 mL), the organic layers combined, dried with anhydrous Na₂SO₄, filtered, and concentrated in vacuo. Yield: 2.98 g, pale yellow oil (85%). ¹H NMR (400 MHz, CDCl₃): δ=1.54 (m, 4H), 2.48 (m, 6H), 2.70 (t, 4H), 3.53 (t, 2H). ¹³C NMR (100 MHz, CDCl₃): δ=30.58, 40.37, 52.01, 56.02, 59.77. Calcd: [M+H]⁺ (C₈H₂₁N₃O) m/z=175.05. Found: CI-MS: [M+H]⁺ m/z=176.2. Anal. Calcd for C₈H₂₁ N₃O: C, 54.86; H, 12.00; N, 24.00%. Found: C, 53.47; H, 12.06; N, 23.67%.

[8] was prepared using the literature procedure: Soft Matter, 2012, 8, 1096.

1.3 Generation 2 (G₂) dendron ROP initiator synthesis

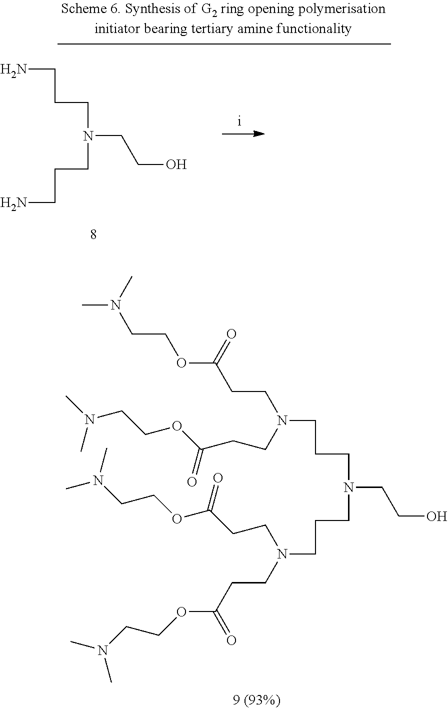 [SMILES: NCCCN(CCO)CCCN ; CN(C)CCOC(=O)CCN(CCCN(CCO)CCCN(CCC(=O)OCCN(C)C)CCC(=O)OCCN(C)C)CCC(=O)OCCN(C)C]

[G₂ dendron ROP initiator]; [9]—DMEA (6.0 g, 0.042 mol, 6 eq.) was added to a 50 mL round 2 necked round-bottomed flask containing IPA (12 mL). The flask was deoxygenated under a positive N₂ purge for 10 minutes. [8] (1.2222 g, 0.007 mmol, 1 eq.) dissolved in IPA (12 mL) was added drop wise while the solution was stirring in an ice bath under a positive flow of N₂. The final mixture was stirred for a further 10 minutes at 0° C., allowed to warm to room temperature and left stirring for 48 hr. The solvent was removed and the product left to dry in vacuo overnight. Yield: 4.84 g, yellow oil, (93%). ¹H NMR (400 MHz, CDCl₃) δ 1.52 (m, 4H), 2.23 (s, 24H), 2.41 (m, 16H). 2.51 (t, 10H), 2.72 (t, 8H), 3.50 (t, 2H), 4.11 (t, 8H). ¹³C NMR (100 MHz, CDCl3) δ 24.62, 32.24, 45.68, 48.94, 51.53, 51.89, 55.76, 57.82, 59.48, 62.15, 172.6. Calcd [M+H]⁺ (C₃₆H₇₃O₉N₇) m/z=748.01. Found: ESI-MS: [M+H]⁺ m/z=748.6. Anal. Calcd for C₃₆H₇₃O₉N₇: C, 57.75; H, 9.76; N, 13.10%. Found: C, 57.50; H, 9.76; N, 13.01%.

[9] was prepared using the literature procedure: Polymer Chemistry, 2015, 6, 573

2.0 4,4′-bioxepanyl-7,7′-dione (BOD) synthesis

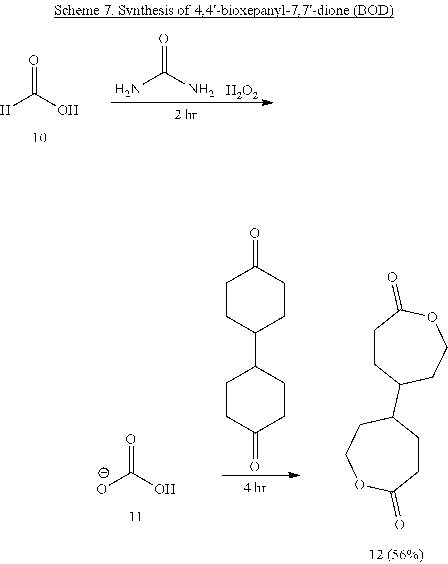

[BOD]; [12]—Urea hydrogen peroxide (10 g, 0.106 mol, 25 eq.) was added to a 250 mL RBF containing formic acid [10] (100 mL, 2.65 mol, 1 eq.). The solution was stirred for 2 hr at room temperature. The flask was then immersed in an ice bath and had bicyclohexanone (10 g, 0.026 mol, 100 eq.) was slowly added to the solution. The reaction mixture was stirred for 4 hr. Water (100 mL) was then added to the mixture and the product was extracted with chloroform (3×100 mL). The organic fractions were collected and washed with saturated aqueous sodium bicarbonate solution (100 mL) then dried over night with Na₂SO₄. After removing the solvent, a white powder was isolated and dried under vacuum overnight. Yield: 3.26 g, white solid, (56%). ¹H NMR (400 MHz, CDCl₃) δ 1.50 (m, 2H), 1.66 (m, 4H), 1.87 (m, 4H), 2.51-2.82 (d of t, 4H), 4.08-4.44 (d of t, 4H). ¹³C NMR (100 MHz, CDCl3) δ 29.6, 35.9, 37.1, 49.7, 72.4, 181.3. Calcd [M+H]⁺ (C₁₂H₁₈O₄) m/z=226.3. Found: ESI-MS: [M+H]⁺ m/z=228.2. Anal. Calcd for C₁₂H₁₈O₄: C, 63.64; H, 7.96%. Found: C, 61.67; H, 7.72%.

[12] was prepared using the literature procedure: Polymer Chemistry, 5 (8), 2997-3008

3.0 Ring Opening Polymerisation of ε-caprolactone

3.1 Typical Polymerisation of ε-caprolactone (CL)

[General procedure for Bn- and G₀-p(CLₓ)]; In a typical experiment, Sn(oct)₂ (0.006 g, 0.0014 mmol, 1/350 eq.) was added using a dry syringe to a RBF equipped with a magnetic stirrer bar flushed with dry nitrogen. Following this, CL (17.75 g, 16.5 mL, 0.16 mol, 30 eq.) was added using a dry syringe. The reaction mixture was degassed for a further 15 minutes and then immersed in a silicon oil bath at 110° C. 2-dimethylaminoethanol (G₀ dendron ROP initiator) (0.4621 g, 0.52 mL, 0.005 mol, 1 eq.) was added via a dry syringe and the polymerisation left for 20 hr. The polymerisation was stopped by removing the reaction mixture from the heat and immersing it in an ice bath. The crude product was dissolved in 50 mL of tetrahydrofuran (THF) and precipitated from 600 mL of hexane. The precipitated polymer was dried under vacuum for 24 hr.

[General procedure for G₁- and G₂-p(CLₓ)]; In a typical experiment, Sn(oct)₂ (0.0055 g, 0.014 mmol, 1/150 eq.) and G₁ dendron ROP initiator [2] (0.7066 g, 0.002 mol, 1 eq.) were added to a RBF equipped with a magnetic stirrer bar flushed with dry nitrogen. The reaction mixture was degassed for a further 15 minutes and then immersed in a silicon oil bath at 110° C. Following this, CL (6.97 g, 6.5 mL, 0.06 mol, 30 eq.) was added using a dry syringe and the polymerisation left for 48 hr. The polymerisation was stopped by removing the reaction mixture from the heat and immersing it in an ice bath. The crude product was dissolved in 50 mL of THF and precipitated from 600 mL of hexane. The precipitated polymer was dried under vacuum for 24 hr.

Polymers were prepared using literature procedures: Macromolecules 1997, 30, 8508, Chem. Comm., 2006, 4010 and Macromolecules, 1998, 31, 2756.

TABLE 1
SEC analysis of linear benzyl-functional polymer and amine-functional
linear-dendritic hybrids

| | [I]/ | Theoretical | Mn (Da) | Mw (Da) | |
|---|---|---|---|---|---|
| Polymer | [catalyst | Mn (Da) | (GPC)^{a} | (GPC)^{a} | Mw/Mn |
| Bn-p(CL₃₀) | 350 | 3,420 | 5,420 | 7,480 | 1.38 |
| G₀-p(CL₂₀) | 350 | 2,280 | 1,790 | 2,520 | 1.41 |
| G₀-p(CL₃₀) | 350 | 3,420 | 2,780 | 3,950 | 1.42 |
| G₀-p(CL₅₀) | 350 | 5,710 | 4,820 | 6,665 | 1.38 |
| G₁-p(CL₂₀) | 200 | 2,280 | 2,220 | 3,754 | 1.70 |
| G₁-p(CL₃₀) | 350 | 3,420 | 3,090 | 6,050 | 1.96 |
| G₁-p(CL₅₀) | 150 | 5,710 | 6,650 | 9,640 | 1.45 |
| G₂-p(CL₃₀) | 350 | 3,420 | 5,210 | 11,460 | 2.20 |

^{a}Triple detection analysis using THF/2% TEA as eluent

3.2 Typical polymerisation of CL and 4,4′-bioxepanyl-7,7′-dione (BOD)

[General procedure for Bn- and G₀-p(CL₃₀-co-BODₓ)]; In a typical experiment, Sn(oct)₂(0.002 g, 0.005 mmol, 1/350 eq.) and BOD (0.3128 g, 0.0014 mol, 0.8 eq.) were added to a RBF equipped with a magnetic stirrer bar flushed with dry nitrogen. Following this, CL (5.9 g, 5.5 mL, 0.052 mol, 30 eq.) was added using a dry syringe. The reaction mixture was degassed for a further 15 minutes and then immersed in a silicon oil bath at 110° C. G₀ dendron ROP initiator (0.15 g, 0.17 mL, 0.0017 mol, 1 eq.) was added via a dry syringe and the polymerisation left for 20 hr. The polymerisation was stopped by removing the reaction mixture from the heat and immersing it in an ice bath. The crude product was dissolved in 50 mL of THF and precipitated from 600 mL of hexane. The precipitated polymer was dried under vacuum for 24 hr.

[General procedure for G₁- and G₂-p(CL₃₀-co-BODₓ)]; In a typical experiment, Sn(oct)₂(0.0032 g, 0.007 mmol, 1/200 eq.), BOD (0.2849 g, 0.0013 mol, 0.8 eq.) and [2] (0.5469 g, 0.0016 mol, 1 eq.) were added to a RBF equipped with a magnetic stirrer bar flushed with dry nitrogen. The reaction mixture was degassed for a further 15 minutes and then immersed in a silicon oil bath at 110° C. Following this, CL (5.4 g, 5 mL, 0.047 mol, 30 eq.) was added using a dry syringe and the polymerisation left for 14 hr. The polymerisation was stopped by removing the reaction mixture from the heat and immersing it in an ice bath. The crude product was dissolved in 30 mL of THF and precipitated from 600 mL of hexane. The precipitated polymer was dried under vacuum for 24 hr.

TABLE 2
SEC analysis of hyperbranched benzyl-functional polymer and
hyperbranched amine-functional polydendrons

| | Mn (Da) | Mw (Da) | |
|---|---|---|---|
| Polymer | (GPC)^{a} | (GPC)^{a} | Mw/Mn |
| Bn-p(CL₃₀-co-BOD_{1.0}) | 5,600 | 108,560 | 19.39 |
| G₀-p(CL₃₀-co-BOD_{1.0}) | 9,480 | 129,510 | 13.67 |
| G₁-p(CL₃₀-co-BOD_{0.8}) | 3,250 | 92,580 | 28.50 |
| G₂-p(CL₃₀-co-BOD_{1.0}) | | | |

^{a}Triple detection analysis using THF/2% TEA as eluent

4.0 Degradation Studies

Polymers for degradation experiments were prepared in a phosphate buffered saline (PBS) solution (0.02 mol L⁻¹, pH=7.4). Each polymer (1 g) was placed into a capped vial containing 20 mL PBS (0.02 mol L⁻¹, pH=7.4). The vial was then left standing at room temperature. At predetermined time intervals, samples were withdrawn (1 mL), frozen in liquid nitrogen and lyophilised for 24 hours. They were then dissolved in a THF/2 v/v% TEA eluent system and analysed by GPC. Degradation studies were carried out following the literature procedure: Polymer Chem. 2014, 5 (13), 4002

FIG. 2 shows an SEC chromatogram of:
Bn-p{CL₃₀) (dotted trace)
Bn-p(CL₃₀) 4 weeks in PBS (solid bold trace)
Bn-p(CL₃₀) 6 weeks in PBS (solid faint trace) and
Bn-p(CL₃₀) 8 weeks in PBS (dashed trace)

5.0 Nanoparticle Formation

[General procedure for aqueous nanoprecipitation of p(CL₃₀) and p(CL₃₀-co-BODₓ)]-The materials were dissolved in THF at a concentration of 5 mg mL⁻¹. 2 mL of this solution was then subjected to a rapid solvent switch through drop wise addition into 10 mL of water, to give a final polymer concentration of 1 mg mL⁻¹ in water after THF removal by evaporation overnight.

TABLE 3
DLS analysis of nanoprecipitated particles from linear-dendritic polymer
hybrids
Nanoprecipitated into water pH = 4

| Polymer | D_{z}(nm)^{a} | PDI | ζ (mV)^{b} |
|---|---|---|---|
| Bn-p(CL₃₀) | — | — | — |
| G₀-p(CL₃₀) | 123 | 0.140 | +84 |
| G₀-p(CL₅₀) | 97 | 0.184 | +51 |
| G₁-p(CL₃₀) | 130 | 0.259 | +66 |
| G₁-p(CL₅₀) | 122, | 0.148 | +62 |
| G₂-p(CL₃₀) | | | |

^{a}All diameters are given as z-average values as measured by dynamic light scattering.
^{b}All zeta potentials are given as surface charge values as measured by dynamic light scattering.

TABLE 4
DLS analysis of nanoprecipitated particles from hyperbranched
polydendrons

| | pH of water | | | | | |
|---|---|---|---|---|---|---|
| | pH = 4 | | | pH = 7.8 | | |
| Polymer | D_{z} (nm)^{a} | PDI | ζ (mV)^{b} | D_{z} (nm)^{a} | PDI | ζ (mV)^{b} |
| Bn-p (CL₃₀-co- | — | — | — | | | |
| BOD_{1.0}) | | | | | | |
| G₀-p (CL₃₀-co- | 145 | 0.140 | +53 | | | |
| BOD_{1.0}) | | | | | | |
| G₁-p (CL₃₀- | 137 | 0.185 | +48 | 151 | 0.081 | +30 |
| co-BOD_{0.8}) | | | | | | |
| G₂-p (CL₃₀-co- | | | | | | |
| BOD_{1.0}), | | | | | | |

^{a}All diameters are given as z-average values as measured by dynamic light scattering.
^{b}All zeta potentials are given as surface charge values as measured by dynamic light scattering.

6.0 Stability Testing

[General procedure for stability testing of p(CL₃₀-co-BODₓ)]-NaOH (0.14 M) (1 mL) was added to the NP dispersion (1 mg mL⁻¹) (10 mL) prepared in water at pH=7.8.

TABLE 5
DLS analysis of nanoprecipitated particles from hyperbranched
polydendrons before and after salt addition

| | pH = 7.8 | | | + salt^{a} | | |
|---|---|---|---|---|---|---|
| Polymer | D_{z} (nm)^{b} | PDI | ζ (mV)^{c} | D_{z} (nm)^{b} | PDI | ζ (mV)^{c} |
| Bn-p (CL₃₀-co- | | | | | | |
| BOD_{1.0}) | | | | | | |
| G₀-p (CL₃₀-co- | | | | | | |
| BOD_{1.0}) | | | | | | |
| G₁-p (CL₃₀-co- | 147 | 0.077 | +35 | 148 | 0.078 | +35 |
| BOD_{0.8}) | | | | | | |
| G₂-p (CL₃₀-co- | | | | | | |
| BOD_{1.0}), | | | | | | |

^{a}Addition of NaOH (0.14M) (1 mL) to NP dispersion (1 mg mL⁻¹) (10 mL)
^{b}All diameters are given as z-average values as measured by dynamic light scattering.
^{c}All zeta potentials are given as surface charge values as measured by dynamic light scattering.

7.0 Fluoresceinamine Encapsulation

[General procedure for fluoresceinamine encapsulation of p(CL₃₀-co-BOD.)]—Fluoresceinamine was dissolved in THF at a concentration of 1 mg mL⁻¹. 1 mL of this solution, along with 2 mL of the polymer solution (5 mg mL⁻¹), was then subjected to a rapid solvent switch through drop wise addition into 10 mL of water, to give a final polymer concentration of 1 mg mL⁻¹, and fluoresceinamine concentration of 0.1 mg mL⁻¹ (10 wt % loading) in water after THF removal by evaporation overnight.

TABLE 6
DLS analysis of fluoresceinamine encapsulated nanoprecipitated particles
from hyperbranched polydendrons

| Polymer | D_{z}(nm)^{a} | PDI |
|---|---|---|
| Bn-p(CL₃₀-co-BOD_{1.0}) | | |
| G₀-p(CL₃₀-co-BOD_{1.0}) | | |
| G₁-p(CL₃₀-co-BOD_{0.8}) | 85 | 0.167 |
| G₂-p(CL₃₀-co-BOD_{1.0}), | | |

^{a}All diameters are given as z-average values as measured by dynamic light scattering.

B. Ring Opening Polymerisations using Lactide and other Components

Analogous polymerisations using lactide, in place of and in addition to caprolactone, were also carried out, with OH-containing initiators and acid catalysis. ROP of lactide yielded polylactide linear polymers. ROP of lactide with caprolactone yielded polycaprolactone-polylactide copolymers. ROP of lactide with BOD yielded branched polylactides. Similarly to branched polymer cores based on caprolactone, branched polymers based on lactide are also effective; they can form soluble, high molecular weight materials, and can be nanoprecipitated to form nanoparticles.

1. A product which is a branched polyester carrying dendrons.

2. A product as claimed in claim 1 comprising polyester chains linked by branches, wherein the polyester chains have between 1 and 6 carbon atoms between ester linkages.

3. A product as claimed in claim 2 wherein the polyester chains have 5 carbon atoms between ester linkages.

4. A product as claimed in claim 2 wherein the polyester chains have 1 carbon atom between ester linkages.

5. A product as claimed in any preceding claim wherein each branch is a single covalent bond.

6. A product as claimed in any of claims 1 to 4 wherein each branch comprises between 1 and 6 carbon atoms.

7. A product as claimed in any preceding claim which is non-gelled.

8. A product as claimed in any preceding claim having on average one branch or fewer per polyester chain.

9. A product as claimed in any preceding claim wherein the dendrons are present at the ends of polyester chains.

10. A product which is a branched polymer scaffold carrying moieties, wherein the branched polymer scaffold comprises polyester, and wherein the moieties comprise a dendron.

11. A product as claimed in claim 10, having feature(s) as defined in any of claims 2 to 9.

12. A product as claimed in claim 10 or claim 11 wherein the branched polymer scaffold comprises not only polyester but also other polymer.

13. A product as claimed in any of claims 10 to 12 wherein the moieties comprise not only a dendron but also one or more further moiety.

14. A product as claimed in claim 13 wherein a further moiety is a further dendron.

15. A product as claimed in claim 13 or claim 14 wherein a further moiety is not a dendron.

16. A product as claimed in claim 15 wherein the moiety which is not a dendron comprises a PEG group.

17. A method of preparing a branched polyester carrying dendrons, comprising ring-opening polymerisation (ROP) of a monofunctional lactone monomer and a difunctional lactone monomer, using a dendron initiator.

18. A method as claimed in claim 17 wherein the monofunctional lactone monomer is caprolactone, or a mixture of caprolactone and another monofunctional lactone monomer.

19. A method as claimed in claim 17 wherein the monofunctional lactone monomer is lactide, glycolide, or a mixture of lactide and glycolide.

20. A method as claimed in any of claims 17 to 19 wherein the difunctional lactone monomer is BOD (4,4′-bioxepanyl-7-7′-dione).

21. A method as claimed in any of claims 17 to 20 wherein the ROP is metal-catalysed.

22. A method as claimed in any of claims 17 to 20 wherein the ROP is acid-catalysed cationic ROP.

23. A method of preparing a branched polymer scaffold carrying dendrons and optionally other moieties, comprising ring-opening polymerisation (ROP) of lactone using a dendron initiator.

24. A method as claimed in claim 23, having feature(s) as defined in any of claims 17 to 22.

25. A method as claimed in claim 23 or claim 24 comprising not only ROP but also other polymerisation.

26. A method as claimed in any of claims 23 to 25 using not only one dendron initiator but also one or more further initiator.

27. A method as claimed in claim 26 wherein a further initiator is a further dendron initiator.

28. A method as claimed in claim 26 or claim 27 wherein a further initiator is not a dendron initiator.

29. A method as claimed in claim 28 wherein the initiator which is not a dendron initiator comprises a PEG group.

30. A product obtainable by the method of any of claims 17 to 29.

31. Use of a product as claimed in any of claim 1 to 16 or 30 in encapsulation.

32. A pharmaceutical composition comprising a product as claimed in any of claim 1 to 16 or 30.

33. A product as claimed in any of claim 1 to 16 or 30, loaded with a therapeutically active material, for use in therapy.

34. A method of medical treatment comprising administration of a product as claimed in any of claim 1 to 16 or 30, loaded with a therapeutically active material, to a subject.
